(19) United States
(12) Patent Application Publication
(10) Pub. No.: US 2011/0046002 A1
(43) Pub. Date: Feb. 24, 2011

(54) Seven Gene Breast Cancer Predictor

(75) Inventors: Timothy Yeatman, Thonotosassa, FL (US); Dung-Tsa Chen, Tampa, FL (US); Aejaz Nasir, Tampa, FL (US)

Correspondence Address: SMITH HOPEN, PA, 180 PINE AVENUE NORTH, OLDSMAR, FL, 34677, US

(73) Assignee: UNIVERSITY OF SOUTH FLORIDA, Tampa, FL (US)

(21) Appl. No.: 12/744,027

(22) PCT Filed: Nov. 20, 2008
(86) PCT No.: PCT/US08/84213

§ 371 Date: Nov. 3, 2010

Related U.S. Application Data
(60) Provisional application No. 60/989,316, filed on Nov. 20, 2007.

(51) Int. Cl. C40B 30/04; C40B 40/08; G01N 33/68

(52) U.S. Cl. 506/9; 506/17; 435/7.1

(57) ABSTRACT

The invention provides a molecular marker set that can be used for prognosis of breast cancer in a patient using histologically normal tissue. The invention also provides methods for evaluating prognosis of breast cancer in a patient based on a molecular molecular signature.

### CROSS REFERENCE TO RELATED APPLICATIONS

This application is a National Stage Application, submitted under 35 U.S.C. §371, of co-pending International Application No. PCT/US08/84213, filed Nov. 20, 2008, which claims priority to and incorporates by reference U.S. Provisional Application No. 60/989,316, filed Nov. 20, 2007, which applications are incorporated herein by reference.

### STATEMENT OF GOVERNMENT INTEREST

This invention was made with Government support under Grant No. CA112215 awarded by the National Institutes of Health. Therefore, the United States Government has rights in the invention.

### BACKGROUND OF THE INVENTION

This invention relates to the field of cancer therapy. Specifically, the invention relates to the field of predicting, and providing prognosis for, breast cancer.

Breast cancer is the second common cancer among American women. The chance of developing invasive breast cancer at some time in a woman's life is about 1 in 8 (12%). Preventive mastectomy is one approach for a woman to reduce risk. Historical data, however, shows a 40% local recurrence rate after lumpectomy without radiotherapy. The rate of local recurrence after mastectomy has been reported at 10-30%, even where the vast majority of normal breast tissue is extirpated. The in-breast local recurrence rate following lumpectomy without radiotherapy for ductal carcinoma in situ is as high as 63% with invasive cancer occurring in over 36% of cases. The median time to recurrence ranges between 2 and 6 years depending on the initial stage of the resected tumor. Radiotherapy significantly reduces rate of local recurrence to 10% or less, but does not eliminate the risk of cancer.

These data emphasize the insensitivity of current strategies to detect disease at an early stage, even in patients known to be high-risk. It is estimated that false negatives and new cancers, previously screened as negative, may amount to 2-4% of new cancer cases. Moreover, there is some evidence for the existence of specific genetic alterations in histologically normal breast tissue adjacent to invasive cancer. For example, Deng et al. reported the loss of heterozygosity (LOH) in histologically normal breast lobules adjacent to a cancer. Eight cases (26.7% from 30 cases) had LOH detected in adjacent morphologically normal tissue. The same allele was missing in the adjacent carcinoma in all these 8 cases. (Science 20 Dec. 1996: Vol. 274. no. 5295, pp. 2057-2059, which is incorporated herein by reference).

The need exists, therefore, a molecular signature for use in screening applications. The ideal molecular signature would be able to detect/predict the occurrence of breast cancer in patients who have not yet been diagnosed with breast cancer. Therefore, the molecular signature should be present in histologically normal breast tissue. The molecular markers comprising the signature could therefore be applied to both normal tissue as well as the normal margins of resected specimens and provide a basis for increasing sensitivity to guide treatment choices.

### SUMMARY OF INVENTION

The invention, as described herein, fulfills the unmet need for a molecular signature for use in screening applications. Sensitivity is increased by detecting specific genetic alterations in histologically normal breast tissue. The histologically normal breast tissue can be taken from a patient who has not been diagnosed with breast cancer or from tissue adjacent to resected invasive cancer.

The invention therefore includes, in a first embodiment, a method for determining a prognosis of breast cancer in a patient by classifying said patient as having a good prognosis or a poor prognosis using measurements of a plurality of gene products in a histologically normal cell sample taken from said patient, said gene products being respectively products of the genes listed in Table 2or respective functional equivalents thereof. Said good prognosis predicts survival of, and/or a lack of the occurrence of breast cancer in, the patient within a predetermined time period from obtaining the sample of histologically normal breast tissue. Said poor prognosis predicts non-survival of, and/or a lack of the occurrence of breast cancer in, a patient within said time period. In a specific embodiment, the predetermined time period is not 2 years. For example, in one embodiment, the predetermined time period is longer than 2 years. In other embodiments, the time period is 4 or 5 years or is between 2 and 6 years.

The invention also includes a method for evaluating whether a breast cancer patient should be treated with chemotherapy by classifying said patient as having a good prognosis or a poor prognosis using a method of the invention and determining that said patient's predicted survival time favors treatment of the patient with adjuvant therapy if said patient is classified as having a poor prognosis.

Furthermore, the invention includes a microarray comprising for each of the plurality of genes listed in Table 2, one or more polynucleotide probes complementary and hybridizable to a sequence in said gene, wherein polynucleotide probes complementary and hybridizable to said genes constitute at least 50% of the probes on said microarray. In one embodiment, the invention provides a kit comprising the microarray of the invention in a sealed container.

### BRIEF DESCRIPTION OF THE DRAWINGS

For a fuller understanding of the invention, reference should be made to the following detailed description, taken in connection with the accompanying drawings, in which:

FIG. 1 Percentile rank distribution of one outlier normal tissue (and one non-outlier normal tissue as a counter example) at various fold-change cutoffs of IDC genes. IDC genes were classified into two categories: up- and down-regulation. The up-regulated genes were displayed in plot of the first column and the down-regulated genes were in plots of the 2nd column. At each plot, Y axis represented the tissue percentile rank. X axis had three fold-change cutoffs: 2, 3, and 4. Number of IDC genes (up- or down-regulated) with a fold-change higher than a cutoff was displayed on the top of the plot (e.g., there were 528 up-regulated IDC genes with a fold greater than 2). Each boxplot displayed distribution of a tissue percentile rank at a specific fold cutoff. For example, the 1st boxplot in the 1st plot was an outlier tissue percentile rank. For each gene from the 528 genes (fold>2), we ranked this outlier tissue among all the HNB tissues to obtain the percentile rank. At the end, there were 528 percentile ranks (corresponding to the 528 genes) to indicate the position of this outlier tissue compared to the rest HNB tissues. In this boxplot (the 1st boxplot), it showed the median of percentile rank for the outlier tissue was beyond 90%. Plots in the 1st row displayed distribution of percentile rank for the outlier tissue. For up-regulated genes in the 1st plot, median of percentile rank was beyond 90% at different fold cutoffs. On the other hand, median of percentile rank was below 20% for the down-regulated genes in the 2nd plot. The results showed this outlier tissue had higher expression (up or down) than the other normal tissues. In contrast, the non-outlier tissue gave a different pattern in the plots of the 2nd row. Median of percentile rank was around 40% and 60% for up- and down-regulated genes, respectively.

FIG. 2 Distribution of median percentile rank among all normal breast tissues.

FIG. 3 Comparison of the outlier gene expression between the outlier normal tissue versus normal breast and IDC tissues based on two-sample t-test. Distribution of p value was displayed in two ways: unadjusted p value (labeled as raw_P) from the two-sample t-test and the adjusted p value based on Benjamini's false discovery rate approach (labeled as fdr_P).

FIG. 4 Principal component analysis of outlier genes in leave-one-out cross validation (LOOCV). At each LOOCV step, the predicted 1st PC score of outlier genes was calculated for the deleted tissue. The plot displayed distribution of the predicted 1st PC score among the three groups: HNB, OBT, to IDC. Result showed an increasing pattern from HNB, OBT, to IDC.

FIG. 5A Among the normal breast tissues, the median of the first PCA score was highest in the outlier tissues followed by the adjacent normal tissues. The other normal tissues (normal reference) had the lowest score. Interestingly, other adjacent tissues, such as cystic change, ALH, and IDC, had also a higher first PCA score compared to the normal reference. Note that the two adjacent IDCs were relatively higher than the other IDCs.

FIG. 5B. The majority of the outlier tissues had a higher score than its adjacent normal or cystic change tissues. One outlier tissue (N11451A4) had a lower score than its adjacent normal tissue. Another outlier tissue (N11103G2) had a higher score than its adjacent normal tissue, but was relatively lower than the other normal tissue. Both outlier tissues were classified because they had a lower median percentile rank less than 20% (i.e., the bottom 20%) for the under-expressed probe sets. However, their median percentile rank for the up-regulated genes was also low (18% and 31%) which might explain a lower PCA score.

FIG. 6 Results showing the PCA score was higher in IDC than in the normal group. The four outlier tissues had a relatively higher score (above 75th percentile) compared to the other normal breast tissues.

FIG. 7 Based on the PCA model derived from the normal breast and IDC tissues (excluding outlier tissues), the inventors calculated the 1st PCA score for the DCSI tissues. Result showed a clear progression pattern from HNB, outlier, DCSI, to IDC.

FIG. 8 Results showing the ADHC group had a higher PCA score than the ADH group. The majority of the ADHC tissues (3 out of 4) yielded a PCA score above 5, in contrast to most AHD tissues with a negative PCA score. This observation indicated the ability of the outlier gene signature to access cancer risk by differentiating AHD tissue between with and without cancer.

FIG. 9 For validation purpose, the inventors used the built-up PCA model from Example IV to predict the 1st PCA score for the 5 IDCs and the associated 10 normal breast tissues. The PCA score was higher in IDC than in normal tissue within the same patient (p value=0.029 based on the random effect model to control for subject variation). This result indicated the outlier gene signature was able to differentiate IDC versus normal tissue.

FIGS. 10A-10C A Principal component analysis of the 21 matched unique genes using the first principal component (PC) in data from Ma et al. Plots in the first column displayed distribution of the 1st PC score (y axis) among the three groups: ADH, DCIS (DC), and IDC (ID). The 2nd column plots were 95% confidence interval of pair-wise comparison for the 1st PC score among the three groups with adjusted p value in the right-hand side's y axis. Principal component analysis was performed in three settings: (a) use all 21 genes, (b) use only the 16 genes with increasing pattern of gene expression, and (c) use only the other 5 genes with non-increasing pattern.

FIG. 11 Sixteen outlier genes showing disease progression from ADH to IDC in data from Ma et al.

FIG. 12 Comparison of van't Veer et al. data.

### DETAILED DESCRIPTION OF THE PREFERRED EMBODIMENT

In the following detailed description of the preferred embodiments, reference is made to the accompanying drawings, which form a part hereof, and within which are shown by way of illustration specific embodiments by which the invention may be practiced. It is to be understood that other embodiments may be utilized and structural changes may be made without departing from the scope of the invention.

The invention provides an innovative approach to identify high-risk normal tissue using gene expression. Herein, normal tissue with tumor-like gene expression, and will thus potentially develop into a tumor, is classified as a high-risk normal tissue. High-risk normal tissue, in one embodiment, is detected by identifying tissue whose expression is the same or resembles the expression in tumor tissue. Often this approach fails because not all tumor genes will have higher expression (up or down) in high-risk normal tissue. Some tumor genes may be the by-product of tumor development. In other words, tumor genes do not always pose risk for cancer development. However, the tumor genes identified herein serve as a key feature or precursor for early stage of tumor development. The expression alteration in these genes increases risk for developing cancer. This type of tumor gene, as disclosed herein, are classified as high-risk genes.

Here, the inventors used a two-stage approach to detect the high-risk normal tissues. In the first step, the inventors employed a classical two-sample comparison approach, Statistical Analysis of Microarray (SAM), to find a tumor gene signature. Since the tumor gene signature differentiates the normal tissue from tumor tissue, the inventors used this gene signature as the reference to identify high-risk normal tissue from the normal tissue. In the second step, the inventors applied an outlier tissue (OT) approach to normal tissue to look for a tissue with tumor like gene expression. Specifically, the OT approach ranks all the normal tissues for each gene in the tumor gene signature. While not all tumor genes associate with cancer development, a high risk normal tissue is likely to have majority of tumor genes with higher expression (up or down). The OT approach allows the identification of such high risk normal tissue (outlier normal tissue) with its percentile rank on the top for a majority of up-regulated tumor genes and/or on the bottom for most down-regulated tumor genes. Since, by definition, the outlier normal tissue has the expression higher than in normal tissue, the tissue may have a greater likelihood to developing tumor. Applying the OT approach to the normal tissues, the inventors identified 11 outlier tissues. A careful examination of all the outlier tissues showed that they were histologically normal with no observable indications of cancer development. However, molecular signature of these outlier tissues suggest that these tissue may be at risk for cancer development since their expression profiles of the gene signature appear closer to that of tumor tissue. This information may be very useful in screening patients for early detection and provide an adequate preventive treatment.

While identification of high risk normal tissue is crucial, it is also important to understand the basic mechanism of how the molecular function changes in the high risk normal tissue. By examining these 11 outlier tissues, the inventors found 117 outlier genes showing significant expression in these tissues. Our pathway analysis showed the IDC tumor genes primarily involved cell adhesion and cell cycle processes. In contrast, the outlier genes, a subset of IDC tumor genes, predominantly comprised cell cycle activities. The high proportion of cell cycle in the outlier genes was over-representative compared to the pathways in the IDC tumor genes. It is well established that de-regulated cell proliferation leads to cancer development. One initial step for tumor development may start with de-regulated cell cycle.

Surprisingly, the inventors observed that the outlier genes were highly associated with two proliferation-related pathways: DNA replication and mitosis. Most the genes were up-regulated. This observation matched well the scenario of early stage of cancer development. By taking these results together, this outlier gene signature provides a simple mechanistic perspective on distinguishing the outlier samples in a population of normal tissues. While the primary function of the majority of these genes spans a variety of metabolic processes, it is clear that nearly all of the components are associated with cellular proliferation, a process that should be limited in normal tissues.

As used herein, “histologically normal breast tissue” or “HNB” refers to substantially benign breast tissue. Substantially benign breast tissue may be comprised of tissue showing other benign changes, e.g. the changes outlined in Table 1. “Histologically normal breast tissue” or “HNB” are substantially free from preneoplastic changes such as atypical lobular hyperplasia (ALH), atypical ductal hyperplasia (ADH), lobular carcinoma in-situ (LCIS), ductal carcinoma in-situ (DCIS) or invasive breast carcinoma.

TABLE 1
outlier tissues and adjacent tissues (bold cells represent outlier tissue)

| Sample | | | |
|---|---|---|---|
| Accession | Banker | | |
| Number | Accession | Container | Pathologic diagnosis |
| T8380A1 | 8380 | A1 | Unremarkable breast ducts |
| N8380A2 | 8380 | A2 | Unremarkable breast ducts |
| N8380A3 | 8380 | A3 | Cystic change/mild |
| | | | ductal hyperplasia/other |
| | | | nonproliferative change |
| N8380A5 | 8380 | A5 | Unremarkable breast ducts |
| N8463I1 | 8463 | I1 | Unremarkable breast ducts |
| N8463I2 | 8463 | I2 | Unremarkable breast ducts |
| N8463I3 | 8463 | I3 | Unremarkable breast ducts |
| N8463I4 | 8463 | I4 | Unremarkable breast ducts |
| T8607A1 | 8607 | A1 | Unremarkable breast ducts |
| N8627A2 | 8627 | A2 | Unremarkable breast ducts |
| N8627A4 | 8627 | A4 | Cystic change/mild |
| | | | ductal hyperplasia/other |
| | | | nonproliferative change |
| T10180C1 | 10180 | C1 | Invasive ductal carcinoma |
| N10180C2 | 10180 | C2 | Unremarkable breast ducts |
| N10180C3 | 10180 | C3 | Unremarkable breast ducts |
| N10180C5 | 10180 | C5 | Cystic change/mild |
| | | | ductal hyperplasia/other |
| | | | nonproliferative change |
| T10739D1 | 10739 | D1 | Invasive ductal carcinoma |
| N10739D4 | 10739 | D4 | Unremarkable breast ducts |
| N10739D5 | 10739 | D5 | Cystic change/mild |
| | | | ductal hyperplasia/other |
| | | | nonproliferative change |
| N10910A1 | 10910 | A1 | Cystic change/mild |
| | | | ductal hyperplasia/other |
| | | | nonproliferative change |
| N10910A2 | 10910 | A2 | Unremarkable breast ducts |
| N10910A3 | 10910 | A3 | Unremarkable breast ducts |
| N10910A4 | 10910 | A4 | Unremarkable breast ducts |
| N11103G1 | 11103 | G1 | Unremarkable breast ducts |
| N11103G2 | 11103 | G2 | Unremarkable breast ducts |
| N11103G4 | 11103 | G4 | Unremarkable breast ducts |
| N11123D1 | 11123 | D1 | Cystic change/mild |
| | | | ductal hyperplasia/other |
| | | | nonproliferative change |
| N11123D2 | 11123 | D2 | Unremarkable breast ducts |
| N11123D3 | 11123 | D3 | Unremarkable breast ducts |
| N11123D4 | 11123 | D4 | Unremarkable breast ducts |
| N11451A2 | 11451 | A2 | Unremarkable breast ducts |
| N11451A3 | 11451 | A3 | Atypical lobular hyperplasia/LCIS |
| N11451A4 | 11451 | A4 | Unremarkable breast ducts |

The invention provides markers, i.e., genes, the expression levels of which discriminate between a good prognosis and a poor prognosis for breast cancer. The identities of these markers and the measurements of their respective gene products, e.g., measurements of levels (abundances) of their encoded mRNAs or proteins, can be used by application of a pattern recognition algorithm to develop a prognosis predictor that discriminates between a good and poor prognosis in breast cancer using measurements of such gene products in a sample from a patient. Such molecular markers, the expression levels of which can be used for prognosis of breast cancer in a breast cancer patient, are listed in Table 2, infra. Measurements of gene products of these molecular markers, as well as of their functional equivalents, can be used for prognosis of breast cancer.

TABLE 2
7 genes overlapping with Vant der veer

| Gene.Title | Gene.Symbol |
|---|---|
| kinetochore associated 2 | KNTC2 |
| maternal embryonic leucine zipper | MELK |
| kinase | |
| centromere protein A, 17 kDa | CENPA |
| cyclin E2 | CCNE2 |
| protein regulator of cytokinesis 1 | PRC1 |
| nucleolar and spindle associated | NUSAP1 |
| protein 1 | |
| denticleless homolog (Drosophila) | DTL |

Therefore, in providing one embodiment, the inventors used normal human breast tissue, compared to human breast cancers to identify a 140 gene signature (subject of another patent application forthcoming) predicting risk of breast cancer development in otherwise histologically normal breast tissue. This 140 signature was tested for its overlap with a published 70 gene signature for breast cancer prognosis (Van't Veer, L J; Dai, H; van de Vijver, M J; He, Y D; Hart, A A M; Mao, M; Peterse, H L; van der Kooy, K; Marton, M J; Witteveen, A T; Schreiber, G J; Kerkhoven, R M; Roberts, C; Linsley, P S; Bernards, R; Friend, S H. Gene expression profiling predicts clinical outcome of breast cancer. Nature. 2002; 415:530-535. doi: 10.1038/415530a) and was found to have 7 overlapping genes.

The inventors then tested the capacity for the 7 gene subset of the original Van't der Veer et al signature to predict outcome on both the Vant der Veer training and test sets of public data. Surprisingly, the 7 gene signature was very effective on both the training and test sets (amounting to two independent test sets for the 7 gene signature). The 7 gene signature of this embodiment can more easily be translated to a paraffin based test and because substantially fewer genes are involved. The 7 gene signature was derived from MCC internal data examining risk of normal breast tissue to become malignant and then tested on two independent public breast cancer data sets.

A functional equivalent with respect to a gene, designated as gene A, refers to a gene that encodes a protein or mRNA that at least partially overlaps in physiological function in the cell to that of the protein or mRNA encoded by gene A.

In particular embodiment, therefore, prognosis of breast cancer in a patient is carried out by a method comprising classifying the patient as having a good or poor prognosis based on a profile of measurements (e.g., of the levels) of gene products of (i.e., encoded by) of the genes in Table 2 or in any of Tables 1, 3, 5 and 6 or any subset thereof, or functional equivalents of such genes; or of at least 2%, 5%, 10%, 20%, 30%, 40%, 50%, 60%, 70%, 80%, or 90% of the genes in Table 2 or in any of Tables 1, 3, 5 and 6 or any subset thereof, or functional equivalents of such genes in an appropriate cell sample from the patient, e.g., a histologically normal breast tissue sample obtained from biopsy or after surgical resection. Different subcombination of genes from Table 2 may be used as the marker set to carry out the prognosis methods of the invention. For example, in various embodiments, the markers that are the genes listed in Table 2, 3, 5 or 6 are used. Preferably, the sample is contaminated with less than 50%, 40%, 30%, 20%, or 10% of abnormal cells. Such a profile of measurements is also referred to herein as an “expression profile.”

In a specific embodiment, the classification of the patient as having good or poor prognosis is carried out using measurements of gene products in which all or at least 20%, 30%, 40%, 50%, 60%, 70%, 80%, or 90% of the genes are from Table 2 or any of Tables 3, 5 and 6 or their functional equivalents.

The measurements in the profiles of the gene products that are used can be any suitable measured values representative of the expression levels of the respective genes. The measurement of the expression level of a gene can be direct or indirect, e.g., directly of abundance levels of RNAs or proteins or indirectly, by measuring abundance levels of cDNAs, amplified RNAs or DNAs, proteins, or activity levels of RNAs or proteins, or other molecules (e.g., a metabolite) that are indicative of the foregoing. In one embodiment, the profile comprisies measurements of abundances of the transcripts of the marker genes. The measurement of abundance can be a measurement of the absolute abundance of a gene product. The measurement of abundance can also be a value representative of the absolute abundance, e.g., a normalized abundance value (e.g., an abundance normalized against the abundance of a reference gene product) or an averaged abundance value (e.g., average of abundances obtained at different time points or from different tumor cell samples from the patients, or average of abundances obtained using different probes, etc.), or a combination of both. As an example, the measurement of abundance of a gene transcript can be a value obtained using an Affymetrix® GeneChip® to measure hybridization to the transcript.

The prognostic methods of the invention are equally useful for determining if a patient is at risk for relapse/recurrence. Cancer relapse is a concern relating to a variety of types of cancer. One explanation for cancer recurrence is that patients with relatively early stage disease already have small amounts of cancer spread outside the affected organ that were not removed by surgery. These cancer cells, referred to as micrometastases, cannot typically be detected with currently available tests. The prognostic methods of the invention can be used to identify surgically treated patients likely to experience cancer recurrence so that they can be offered additional therapeutic options, including preoperative or postoperative adjuncts such as chemotherapy, radiation, biological modifiers and other suitable therapies. The methods are especially effective for determining the risk of metastasis in patients who demonstrate no measurable metastasis at the time of examination or surgery.

The prognostic methods of the invention also are useful for determining a proper course of treatment for a patient having cancer. A course of treatment refers to the therapeutic measures taken for a patient after diagnosis or after treatment for cancer. For example, a determination of the likelihood for cancer development, recurrence, spread, or patient survival, can assist in determining whether a more conservative or more radical approach to therapy should be taken, or whether treatment modalities should be combined. For example, when cancer recurrence is likely, it can be advantageous to precede or follow surgical treatment with chemotherapy, radiation, immunotherapy, biological modifier therapy, gene therapy, vaccines, and the like, or adjust the span of time during which the patient is treated.

As used herein, a good prognosis predicts survival of, and/or the lack of cancer development in, a patient within a predetermined time period from surgical removal of tumor, diagnosis of breast cancer, or from the taking of a histologically normal sample. A poor prognosis predicts non-survival of, or the development of cancer in, a patient within the time period. The predetermined time period is preferably 2, 3, 4, 5 or 6 years. In a specific embodiment, the predetermined time period is not 2 years.

Diagnostic and Prognostic Marker Sets

The invention provides molecular marker sets (of genes) that can be used for prognosis of breast cancer in a breast cancer patient based on a profile of the markers in the marker set (containing measurements of marker gene products). Tables 2, 3, 5 and 6 list markers that can be used to discriminate between good and poor prognosis of breast cancer according to the method of the invention.

Genes that are not listed in T Table 2 or in any of Tables 1, 3, 5 and 6 or any subset thereof but which are functional equivalents of any gene listed therein can also be used with or in place of the gene listed in the table. A functional equivalent of a gene A refers to a gene that encodes a protein or mRNA that at least partially overlaps in physiological function in the cell to that of the protein or mRNA of gene A.

In various specific embodiments, different numbers and sub-combinations of the genes listed in Table 2 are selected as the marker set, whose profile is used in the prognostic methods of the invention. In various embodiments, such sub-combinations include but are not limited to those genes listed in Table 2 or in any of Tables 1, 3, 5 and 6 or any subset thereof, as applicable, or their respective functional equivalents.

In a specific embodiment, one or more of the genes listed in Table 2 can be used to subdivide a patient population into subgroups according to the expression levels of such genes, with the prognostic methods of the invention then applied to such a patient subgroup. For example, in a specific embodiment, the prognostic methods of the invention are applied to patients that have a signature expression level higher than a predetermined threshold, e.g., the average level in subjects not having breast cancer or the average level in breast cancer patients.

In one embodiment, a leave-one-out cross-validation method (LOOCV) is used to obtain a marker set using cDNA data of a training population of patients. In another specific embodiment, measurements of products of a set of genes that are selected in about 75% of the training population in a leave-one-out cross-validation (LOOCV) or their respective functional equivalents are used for prognosis according to the invention. In a specific embodiment, cross-platform mapping of marker genes can also be carried out. For example, translation of cDNA gene signature into available Affymetrix® probe sets is carried out using the Resourcerer program (WWW.TIGR.org). In another embodiment, SAM is used to identify a set of genes most correlated with censored survival time.

Methods of Predicting Cancer Outcome

The invention provides methods for predicting prognosis of breast cancer in a breast cancer patient using a measured marker profile comprising measurements of the gene products of genes, e.g., the sets of genes described supra. The prognosis indicates the patient's predicted condition at a predetermined time after the sample is taken, e.g., at 2, 3, 4, 5 or 6 years.

In preferred embodiments, the methods of the invention use a prognosis predictor, also called a classifier, for predicting prognosis. The prognosis predictor can be based on any appropriate pattern recognition method that receives an input comprising a marker profile and provides an output comprising data indicating a good prognosis or a poor prognosis. The prognosis predictor is trained with training data from a training population of breast cancer patients. Typically, the training data comprise for each of the breast cancer patients in the training population a marker profile comprising measurements of respective gene products of a plurality of genes in a histologically normal breast tissue sample taken from the patient and prognosis outcome information. The marker profile can be obtained by measuring the plurality of gene products in a histologically normal breast tissue sample from the patient using a method known in the art.

In a specific embodiment, the prognosis method of the invention can be used for evaluating whether a breast cancer patient may benefit from chemotherapy. Thus, in one embodiment, the invention provides a method for evaluating whether a breast cancer patient should be treated with chemotherapy, comprising (a) classifying said patient as having a good prognosis or a poor prognosis using a method described above; and (b) determining that said patient's predicted survival time favors treatment of the patient with chemotherapy if said patient is classified as having a poor prognosis.

The prognosis method of the invention can also be used in selecting patients for enrollment for a clinical trial of a chemotherapeutic agent for breast cancer. In one embodiment, this can be achieved using a method comprising (a) classifying each patient as having a good prognosis or a poor prognosis using a method described above; and (b) selecting patients having a poor prognosis for the clinical trial. By only enrolling patients having a poor prognosis, the efficacy of the chemotherapeutic agent can be more reliably evaluated. In a specific embodiment, the invention provides a method for enrolling breast cancer patients for a clinical trial of a chemotherapeutic agent for breast cancer, comprising (a) classifying each patient as having a good prognosis or a poor prognosis using a method described above; and (b) assigning each patient having a good prognosis to one patient group and each patient having a poor prognosis to another patient group, at least one of said patient group being enrolled in said clinical trial.

Statistical Methods

Various known statistical pattern recognition methods can be used in conjunction with the present invention. A prognosis predictor based on any of such methods can be constructed using the marker profiles and prognosis data of training patients. Such a prognosis predictor can then be used to predict prognosis of a breast patient based on the patient's marker profile. The methods can also be used to identify markers that discriminate between a good and poor prognosis using a marker profile and prognosis data of training patients.

Determination of Abundance Levels of Gene Products

The abundance levels of the gene products of the genes in a sample may be determined by any means known in the art. The levels may be determined by isolating and determining the level (i.e., amount) of nucleic acid transcribed from each marker gene. Alternatively, or additionally, the level of specific proteins encoded by a marker gene may be determined.

The levels of transcripts of specific marker genes can be accomplished by determining the amount of mRNA, or polynucleotides derived therefrom, present in a sample. Any method for determining RNA levels can be used. For example, RNA is isolated from a sample and separated on an agarose gel. The separated RNA is then transferred to a solid support, such as a filter. Nucleic acid probes representing one or more markers are then hybridized to the filter by northern hybridization, and the amount of marker-derived RNA is determined. Such determination can be visual, or machine-aided, for example, by use of a densitometer. Another method of determining RNA levels is by use of a dot-blot or a slot-blot. In this method, RNA, or nucleic acid derived therefrom, from a sample is labeled. The RNA or nucleic acid derived therefrom is then hybridized to a filter containing oligonucleotides derived from one or more marker genes, wherein the oligonucleotides are placed upon the filter at discrete, easily-identifiable locations. Hybridization, or lack thereof, of the labeled RNA to the filter-bound oligonucleotides is determined visually or by densitometer. Polynucleotides can be labeled using a radiolabel or a fluorescent (i.e., visible) label. These examples are not intended to be limiting; other methods of determining RNA abundance are known in the art.

The levels of transcripts of particular marker genes may also be assessed by determining the level of the specific protein expressed from the marker genes. This can be accomplished, for example, by separation of proteins from a sample on a polyacrylamide gel, followed by identification of specific marker-derived proteins using antibodies in a western blot. Alternatively, proteins can be separated by two-dimensional gel electrophoresis systems. Two-dimensional gel electrophoresis is well-known in the art and typically involves isoelectric focusing along a first dimension followed by SDS-PAGE electrophoresis along a second dimension. See, e.g., Hames et al, 1990, GEL ELECTROPHORESIS OF PROTEINS: A PRACTICAL APPROACH, IRL Press, New York; Shevchenko et al., Proc. Nat'l Acad. Sci. USA 93:1440-1445 (1996); Sagliocco et al., Yeast 12:1519-1533 (1996); Lander, Science 274:536-539 (1996). The resulting electropherograms can be analyzed by numerous techniques, including mass spectrometric techniques, western blotting and immunoblot analysis using polyclonal and monoclonal antibodies.

Alternatively, marker-derived protein levels can be determined by constructing an antibody microarray in which binding sites comprise immobilized, preferably monoclonal, antibodies specific to a plurality of protein species encoded by the cell genome. Preferably, antibodies are present for a substantial fraction of the marker-derived proteins of interest. Methods for making monoclonal antibodies are well known (see, e.g., Harlow and Lane, 1988, ANTIBODIES: A LABORATORY MANUAL, Cold Spring Harbor, N.Y., which is incorporated in its entirety for all purposes). In one embodiment, monoclonal antibodies are raised against synthetic peptide fragments designed based on genomic sequence of the cell. With such an antibody array, proteins from the cell are contacted to the array, and their binding is assayed with assays known in the art. Generally, the expression, and the level of expression, of proteins of diagnostic or prognostic interest can be detected through immunohistochemical staining of tissue slices or sections.

Finally, levels of transcripts of marker genes in a number of tissue specimens may be characterized using a “tissue array” (Kononen et al., Nat. Med 4(7):844-7 (1998)). In a tissue array, multiple tissue samples are assessed on the same microarray. The arrays allow in situ detection of RNA and protein levels; consecutive sections allow the analysis of multiple samples simultaneously.

Microarrays

In preferred embodiments, polynucleotide microarrays are used to measure expression so that the expression status of each of the markers above is assessed simultaneously. Generally, microarrays according to the invention comprise a plurality of markers informative for prognosis, or outcome determination, for a particular disease or condition, and, in particular, for individuals having specific combinations of genotypic or phenotypic characteristics of the disease or condition (i.e., that are prognosis-informative for a particular patient subset).

The invention also provides a microarray comprising for each of a plurality of genes, said genes being all or at least 1, 2, 4 or 6 of the genes listed in Table 2, one or more polynucleotide probes complementary and hybridizable to a sequence in said gene, wherein polynucleotide probes complementary and hybridizable to said genes constitute at least 50%, 60%, 70%, 80%, 90%, 95%, or 98% of the probes on said microarray. In a particular embodiment, the invention provides such a microarray wherein the plurality of genes comprises the genes listed in Table 2. The microarray can be in a sealed container.

The microarrays of the invention preferably comprise at least 2, 3, 4, 5, or 6 of markers, or all of the markers, or any combination of markers, identified as prognosis-informative within a patient subset, e.g., within Table 2. The actual number of informative markers the microarray comprises will vary depending upon the particular condition of interest.

In specific embodiments, the invention provides polynucleotide arrays in which the prognosis markers identified for a particular patient subset comprise at least 50%, 60%, 70%, 80%, 85%, 90%, 95% or 98% of the probes on the array. In another specific embodiment, the microarray comprises a plurality of probes, wherein said plurality of probes comprise probes complementary and hybridizable to at least 75% of the prognosis-informative markers identified for a particular patient subset. Microarrays of the invention, of course, may comprise probes complementary and hybridizable to prognosis-informative markers for a plurality of the patient subsets, or for each patient subset, identified for a particular condition. In another embodiment, therefore, the microarray of the invention comprises a plurality of probes complementary and hybridizable to at least 75% of the prognosis-informative markers identified for each patient subset identified for the condition of interest, and wherein the probes, in total, are at least 50% of the probes on said microarray.

Detection and Quantification of Protein

Measurement of the translational state may be performed according to several methods. For example, whole genome monitoring of protein (e.g., the “proteome,”) can be carried out by constructing a microarray in which binding sites comprise immobilized, preferably monoclonal, antibodies specific to a plurality of protein species encoded by the cell genome. Preferably, antibodies are present for a substantial fraction of the encoded proteins, or at least for those proteins relevant to the action of a drug of interest. Methods for making monoclonal antibodies are well known (see, e.g., Harlow and Lane, 1988, Antibodies: A Laboratory Manual, Cold Spring Harbor, N.Y., which is incorporated in its entirety for all purposes). In one embodiment, monoclonal antibodies are raised against synthetic peptide fragments designed based on genomic sequence of the cell. With such an antibody array, proteins from the cell are contacted to the array and their binding is assayed with assays known in the art.

Immunoassays known to one of skill in the art can be used to detect and quantify protein levels. For example, ELISAs can be used to detect and quantify protein levels. ELISAs comprise preparing antigen, coating the well of a 96 well microtiter plate with the antigen, adding the antibody of interest conjugated to a detectable compound such as an enzymatic substrate (e.g., horseradish peroxidase or alkaline phosphatase) to the well and incubating for a period of time, and detecting the presence of the antigen. In ELISAs the antibody of interest does not have to be conjugated to a detectable compound; instead, a second antibody (which recognizes the antibody of interest) conjugated to a detectable compound may be added to the well. Further, instead of coating the well with the antigen, the antibody may be coated to the well. In this case, a second antibody conjugated to a detectable compound may be added following the addition of the antigen of interest to the coated well. One of skill in the art would be knowledgeable as to the parameters that can be modified to increase the signal detected as well as other variations of ELISAs known in the art. In a preferred embodiment, an ELISA may be performed by coating a high binding 96-well microtiter plate (Costar) with 2 .mu.g/ml of rhu-IL-9 in PBS overnight. Following three washes with PBS, the plate is incubated with three-fold serial dilutions of Fab at 25.degree. C. for 1 hour. Following another three washes of PBS, 1 .mu.g/ml anti-human kappa-alkaline phosphatase-conjugate is added and the plate is incubated for 1 hour at 25.degree. C. Following three washes with PBST, the alkaline phosphatase activity is determined in 50 .mu.l/AMP/PPMP substrate. The reactions are stopped and the absorbance at 560 nm is determined with a VMAX microplate reader. For further discussion regarding ELISAs see, e.g., Ausubel et al, eds, 1994, Current Protocols in Molecular Biology, Vol. 1, John Wiley & Sons, Inc., New York at 11.2.1.

Protein levels may be determined by Western blot analysis. Further, protein levels as well as the phosphorylation of proteins can be determined by immunoprecitation followed by Western blot analysis. Immunoprecipitation protocols generally comprise lysing a population of cells in a lysis buffer such as RIPA buffer (1% NP-40 or Triton X-100, 1% sodium deoxycholate, 0.1% SDS, 0.15 M NaC1, 0.01 M sodium phosphate at pH 7.2, 1% Trasylol) supplemented with protein phosphatase and/or protease inhibitors (e.g., EDTA, PMSF, aprotinin, sodium vanadate), adding the antibody of interest to the cell lysate, incubating for a period of time (e.g., 1 to 4 hours) at 40.degree. C., adding protein A and/or protein G sepharose beads to the cell lysate, incubating for about an hour or more at 40.degree. C., washing the beads in lysis buffer and resuspending the beads in SDS/sample buffer. The ability of the antibody of interest to immunoprecipitate a particular antigen can be assessed by, e.g., western blot analysis. One of skill in the art would be knowledgeable as to the parameters that can be modified to increase the binding of the antibody to an antigen and decrease the background (e.g., pre-clearing the cell lysate with sepharose beads). For further discussion regarding immunoprecipitation protocols see, e.g., Ausubel et al, eds, 1994, Current Protocols in Molecular Biology, Vol. 1, John Wiley & Sons, Inc., New York at 10.16.1.

Western blot analysis generally comprises preparing protein samples, electrophoresis of the protein samples in a polyacrylamide gel (e.g., 8%-20% SDS-PAGE depending on the molecular weight of the antigen), transferring the protein sample from the polyacrylamide gel to a membrane such as nitrocellulose, PVDF or nylon, incubating the membrane in blocking solution (e.g., PBS with 3% BSA or non-fat milk), washing the membrane in washing buffer (e.g., PBS-Tween 20), incubating the membrane with primary antibody (the antibody of interest) diluted in blocking buffer, washing the membrane in washing buffer, incubating the membrane with a secondary antibody (which recognizes the primary antibody, e.g., an anti-human antibody) conjugated to an enzymatic substrate (e.g., horseradish peroxidase or alkaline phosphatase) or radioactive molecule (e.g., .sup.32P or .sup.125I) diluted in blocking buffer, washing the membrane in wash buffer, and detecting the presence of the antigen. One of skill in the art would be knowledgeable as to the parameters that can be modified to increase the signal detected and to reduce the background noise. For further discussion regarding western blot protocols see, e.g., Ausubel et al, eds, 1994, Current Protocols in Molecular Biology, Vol. 1, John Wiley & Sons, Inc., New York at 10.8.1.

Protein expression levels can also be separated by two-dimensional gel electrophoresis systems. Two-dimensional gel electrophoresis is well-known in the art and typically involves iso-electric focusing along a first dimension followed by SDS-PAGE electrophoresis along a second dimension. See, e.g., Hames et al., 1990, Gel Electrophoresis of Proteins: A Practical Approach, IRL Press, New York; Shevchenko et al., 1996, Proc. Natl. Acad. Sci. USA 93:1440-1445; Sagliocco et al., 1996, Yeast 12:1519-1533; Lander, 1996, Science 274:536-539. The resulting electropherograms can be analyzed by numerous techniques, including mass spectrometric techniques, Western blotting and immunoblot analysis using polyclonal and monoclonal antibodies, and internal and N-terminal micro-sequencing.

Determining Therapeutic Regimens for Patients

The methods of the prognosis prediction can be used for determining whether a breast cancer patient may benefit from chemotherapy. In one embodiment, the invention provides a method for determining whether a breast cancer patient should be treated with chemotherapy, comprising (a) classifying the patient as having a good prognosis or a poor prognosis using a method as described above; and (b) determining that said patient's predicted survival time favors treatment of the patient with chemotherapy if said patient is classified as having a poor prognosis.

If a patient is determined to be one likely to benefit from chemotherapy, a suitable chemotherapy may be prescribed for the patient. Chemotherapy can be performed using any one or a combination of the anti-cancer drugs known in the art, including but not limited to any topoisomerase inhibitor, DNA binding agent, anti-metabolite, ionizing radiation, or a combination of two or more of such known DNA damaging agents.

A topoisomerase inhibitor that can be used in conjunction with the invention can be, for example, a topoisomerase I (Topo I) inhibitor, a topoisomerase II (Topo II) inhibitor, or a dual topoisomerase I and II inhibitor. A topo I inhibitor can be from any of the following classes of compounds: camptothecin analogue (e.g., karenitecin, aminocamptothecin, lurtotecan, topotecan, irinotecan, BAY 56-3722, rubitecan, GI14721, exatecan mesylate), rebeccamycin analogue, PNU 166148, rebeccamycin, TAS-103, camptothecin (e.g., camptothecin polyglutamate, camptothecin sodium), intoplicine, ecteinascidin 743, J-107088, pibenzimol. Examples of preferred topo I inhibitors include but are not limited to camptothecin, topotecan (hycaptamine), irinotecan (irinotecan hydrochloride), belotecan, or an analogue or derivative thereof.

A topo II inhibitor that can be used in conjunction with the invention can be, for example, from any of the following classes of compounds: anthracycline antibiotics (e.g., carubicin, pirarubicin, daunorubicin citrate liposomal, daunomycin, 4-iodo-4-doxydoxorubicin, doxorubicin, n,n-dibenzyl daunomycin, morpholinodoxorubicin, aclacinomycin antibiotics, duborimycin, menogaril, nogalamycin, zorubicin, epirubicin, marcellomycin, detorubicin, annamycin, 7-cyanoquinocarcinol, deoxydoxorubicin, idarubicin, GPX-100, MEN-10755, valrubicin, KRN5500), epipodophyllotoxin compound (e.g., podophyllin, teniposide, etoposide, GL331, 2-ethylhydrazide), anthraquinone compound (e.g., ametantrone, bisantrene, mitoxantrone, anthraquinone), ciprofloxacin, acridine carboxamide, amonafide, anthrapyrazole antibiotics (e.g., teloxantrone, sedoxantrone trihydrochloride, piroxantrone, anthrapyrazole, losoxantrone), TAS-103, fostriecin, razoxane, XK469R, XK469, chloroquinoxaline sulfonamide, merbarone, intoplicine, elsamitrucin, CI-921, pyrazoloacridine, elliptinium, amsacrine. Examples of preferred topo II inhibitors include but are not limited to doxorubicin (Adriamycin), etoposide phosphate (etopofos), teniposide, sobuzoxane, or an analogue or derivative thereof.

DNA binding agents that can be used in conjunction with the invention include but are not limited to DNA groove binding agent, e.g., DNA minor groove binding agent; DNA crosslinking agent; intercalating agent; and DNA adduct forming agent. A DNA minor groove binding agent can be an anthracycline antibiotic, mitomycin antibiotic (e.g., porfiromycin, KW-2149, mitomycin B, mitomycin A, mitomycin C), chromomycin A3, carzelesin, actinomycin antibiotic (e.g., cactinomycin, dactinomycin, actinomycin F1), brostallicin, echinomycin, bizelesin, duocarmycin antibiotic (e.g., KW 2189), adozelesin, olivomycin antibiotic, plicamycin, zinostatin, distamycin, MS-247, ecteinascidin 743, amsacrine, anthramycin, and pibenzimol, or an analogue or derivative thereof.

DNA crosslinking agents include but are not limited to antineoplastic alkylating agent, methoxsalen, mitomycin antibiotic, psoralen. An antineoplastic alkylating agent can be a nitrosourea compound (e.g., cystemustine, tauromustine, semustine, PCNU, streptozocin, SarCNU, CGP-6809, carmustine, fotemustine, methylnitrosourea, nimustine, ranimustine, ethylnitrosourea, lomustine, chlorozotocin), mustard agent (e.g., nitrogen mustard compound, such as spiromustine, trofosfamide, chlorambucil, estramustine, 2,2,2-trichlorotriethylamine, prednimustine, novembichin, phenamet, glufosfamide, peptichemio, ifosfamide, defosfamide, nitrogen mustard, phenesterin, mannomustine, cyclophosphamide, melphalan, perfosfamide, mechlorethamine oxide hydrochloride, uracil mustard, bestrabucil, DHEA mustard, tallimustine, mafosfamide, aniline mustard, chlomaphazine; sulfur mustard compound, such as bischloroethylsulfide; mustard prodrug, such as TLK286 and ZD2767), ethylenimine compound (e.g., mitomycin antibiotic, ethylenimine, uredepa, thiotepa, diaziquone, hexamethylene bisacetamide, pentamethylmelamine, altretamine, carzinophilin, triaziquone, meturedepa, benzodepa, carboquone), alkylsulfonate compound (e.g., dimethylbusulfan, Yoshi-864, improsulfan, piposulfan, treosulfan, busulfan, hepsulfam), epoxide compound (e.g., anaxirone, mitolactol, dianhydrogalactitol, teroxirone), miscellaneous alkylating agent (e.g., ipomeanol, carzelesin, methylene dimethane sulfonate, mitobronitol, bizelesin, adozelesin, piperazinedione, VNP40101M, asaley, 6-hydroxymethylacylfulvene, EO9, etoglucid, ecteinascidin 743, pipobroman), platinum compound (e.g., ZD0473, liposomal-cisplatin analogue, satraplatin, BBR 3464, spiroplatin, ormaplatin, cisplatin, oxaliplatin, carboplatin, lobaplatin, zeniplatin, iproplatin), triazene compound (e.g., imidazole mustard, CB 10-277, mitozolomide, temozolomide, procarbazine, dacarbazine), picoline compound (e.g., penclomedine), or an analogue or derivative thereof. Examples of preferred alkylating agents include but are not limited to cisplatin, dibromodulcitol, fotemustine, ifosfamide (ifosfamid), ranimustine (ranomustine), nedaplatin (latoplatin), bendamustine (bendamustine hydrochloride), eptaplatin, temozolomide (methazolastone), carboplatin, altretamine (hexamethylmelamine), prednimustine, oxaliplatin (oxalaplatinum), carmustine, thiotepa, leusulfon (busulfan), lobaplatin, cyclophosphamide, bisulfan, melphalan, and chlorambucil, or analogues or derivatives thereof.

Intercalating agents can be an anthraquinone compound, bleomycin antibiotic, rebeccamycin analogue, acridine, acridine carboxamide, amonafide, rebeccamycin, anthrapyrazole antibiotic, echinomycin, psoralen, LU 79553, BW A773U, crisnatol mesylate, benzo(a)pyrene-7,8-diol-9,10-epoxide, acodazole, elliptinium, pixantrone, or an analogue or derivative thereof, etc.

DNA adduct forming agents include but are not limited to enediyne antitumor antibiotic (e.g., dynemicin A, esperamicin Al, zinostatin, dynemicin, calicheamicin gamma 1I), platinum compound, carmustine, tamoxifen (e.g., 4-hydroxy-tamoxifen), psoralen, pyrazine diazohydroxide, benzo(a)pyrene-7,8-diol-9,10-epoxide, or an analogue or derivative thereof. Anti-metabolites include but are not limited to cytosine, arabinoside, floxuridine, fluorouracil, mercaptopurine, Gemcitabine, and methotrexate (MTX).

### Examples

The invention, as it has been shown, includes a high cancer-risk gene signature in morphologically normal breast tissues obtained from patients with breast cancer. The inventors identified a plurality of outlier genes, with expression similar to invasive ductal carcinomas (IDCs), in normal breast tissues (outlier normal breast tissue) taken from 143 histologically normal samples from patients that underwent mastectomy at various stages of breast carcinoma. The outlier genes are highly associated with cell proliferation.

The resulting outlier gene signature was tested at several external datasets. These external datasets provided various aspects to evaluate the properties of the gene signature, including cross-platform validation, disease progression, and cancer risk (including metastasis). The outlier gene signature derived from the outlier normal breast tissue was further validated by Ma data and revealed a progression pattern from atypical ductal hyperplasia (ADH) to IDC.

Tissues were collected in accordance with the protocols approved by the Institutional Review Board of the University of South Florida, and stored in the tissue bank of Moffitt Cancer Center. Breast tissues from patients that underwent mastectomy at various stages of breast carcinoma were collected. Mastectomy specimens were marked for tumor and its four surrounding zones (1 cm, 2 cm, 3 cm and 4 cm away from the grossly visible tumor boundary). Samples from each zone were collected and frozen in liquid nitrogen. In bilateral and prophylactic mastectomy cases, normal breast tissues were taken from four random sections of the breast. The tissues were embedded in Tissue-Tek® O.C.T., 5-μm sections cut and mounted on Mercedes Platinum StarFrost™ Adhesive slides. The slides were stained using a standard H&E protocol, and tissue boundaries marked. Using the marked slide as a “map”, tissues were microdissected. Adipose tissues were trimmed away; the tumor and “normal” tissues were separated and stored in liquid nitrogen.

Histological examination of all tissue sections and microdissection of samples were conducted in close collaboration with a pathologist to ensure consistency in the clinical diagnoses. The inventors identified a set of 42 histologically invasive ductal carcinomas (IDCs) of various histologic grades (Bloom and Richardson grading). All frozen IDC tissues were reviewed by an experienced breast pathologist for the proportion of IDC component in each frozen tissue specimen that was subjected to RNA extraction for subsequent gene expression profiling. In addition to 42 IDCs, the inventors selected 143 ‘histologically normal breast’ tissues which were confirmed to be free of any other breast lesions upon careful histologic review by the same pathologist.

Total RNA was extracted from breast tissues using the Trizol method (Invitrogen®). Briefly, tissues were ground in liquid nitrogen, resuspended in 5 ml of lysis buffer and incubated for 3 minutes at room temperature and centrifuged at 11,500 g for 15 minutes at 4° C. The aqueous phase was removed and put into another tube with 2.5 ml of isopropanol, mixed well and set at −20° C. for 20 minutes. DNA was pelleted by centrifuging at 11,500 g for 10 minutes at 4° C. The pellet was washed with 75% ethanol and resuspended in 100 μl of deionized water. The amount of RNA was quantitated by measuring A₂₆₀.

### Example I

### Outlier Tissue (OT) Approach

The IDC gene signatures of 42 histologically IDCs and 143 ‘histologically normal breast’ (HNB) tissues were determined by SAM. Analyses of the results showed 43% (of a total of 54,675) probe sets with q value (false discovery rate) less than 0.01. Based on a cutoff of q=0 and a fold change >2, the inventors identified 1,554 probe sets (1038 unique genes) that were differentially expressed between the IDCs and the ‘HNB’ tissues.

Pathway analysis of the IDC signature revealed two predominant cellular processes: cell cycle and cell adhesion (Table 3). There were 10 cell adhesion and 7 cell cycle pathways significantly represented based on a pathway p-value of 0.01. The cell adhesion pathways consisted mostly of intracellular and extracelluar matrix remodeling. Majority of the genes in this category were down-regulated. For cell cycle, many pathways activated are directly involved in of the mitotic phase of the cell cycle.

TABLE 3
Pathway analysis of IDC gene signature with two predominant cellular processes: cell
adhesion and cell cycle

| | | | Number of | Total |
|---|---|---|---|---|
| Cell adhesion | | p- | Genes in IDC | Number of |
| Map | Cell process | Value | gene signature | Genes |
| ECM remodeling | cell adhesion | 1.62E−07 | 17 | 60 |
| Keratin filaments | cell adhesion | 8.06E−06 | 13 | 48 |
| Plasmin signaling | cell adhesion | 1.70E−04 | 11 | 47 |
| Cytoskeleton remodeling | cell adhesion | 4.69E−04 | 24 | 176 |
| Chemokines and | cytokine and chemokine | 3.96E−04 | 24 | 174 |
| adhesion | mediated signaling | | | |
| | pathway, cell adhesion | | | |
| Integrin outside-in | cell adhesion | 1.69E−03 | 13 | 79 |
| signaling | | | | |
| Role of tetraspanins in | cell adhesion | 3.04E−03 | 10 | 56 |
| the integrin-mediated cell | | | | |
| adhesion | | | | |
| Endothelial cell contacts | cell adhesion | 3.77E−03 | 8 | 40 |
| by non-junctional | | | | |
| mechanisms | | | | |
| Cell-matrix | cell adhesion | 4.70E−03 | 7 | 33 |
| glycoconjugates | | | | |
| TGF, WNT and | cell adhesion | 7.25E−03 | 23 | 204 |
| cytoskeletal remodeling | | | | |
| Slit-Robo signaling | cell adhesion | 9.76E−03 | 9 | 56 |
| Angiotensin signaling via | G-protein coupled | 6.79E−03 | 9 | 53 |
| STATs | receptor protein | | | |
| | signaling pathway, | | | |
| | response to extracellular | | | |
| | stimulus | | | |
| FGF-family signaling | intracellular receptor- | 2.44E−04 | 9 | 34 |
| | mediated signaling | | | |
| | pathway, response to | | | |
| | extracellular stimulus | | | |
| PDGF activation of | intracellular receptor- | 4.90E−03 | 5 | 18 |
| prostacyclin synthesis | mediated signaling | | | |
| | pathway, response to | | | |
| | extracellular stimulus | | | |
| The metaphase | cell cycle | 2.24E−09 | 15 | 36 |
| checkpoint | | | | |
| Chromosome | cell cycle | 4.38E−06 | 11 | 33 |
| condensation in | | | | |
| prometaphase | | | | |
| Role APC in cell cycle | cell cycle | 2.59E−05 | 13 | 53 |
| regulation | | | | |
| Nucleocytoplasmic | cell cycle | 3.52E−04 | 7 | 22 |
| transport of CDK/Cyclins | | | | |
| Spindle assembly and | cell cycle | 4.06E−04 | 15 | 86 |
| chromosome separation | | | | |
| Initiation of mitosis | cell cycle | 3.44E−03 | 9 | 48 |
| Sister chromatid cohesion | cell cycle | 6.60E−03 | 7 | 35 |
| Role of Nek in cell cycle | cell cycle, protein kinase | 4.85E−05 | 13 | 56 |
| regulation | cascade | | | |

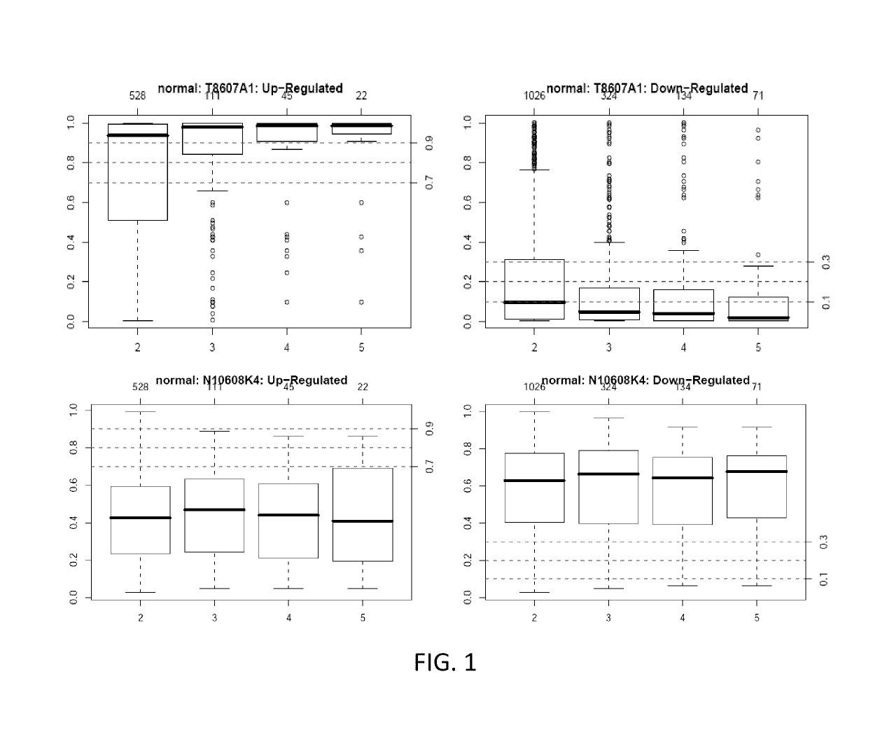

Using the OT approach to the 143 ‘HNB’ tissues, the inventors identified 11 ONTs, whose gene expression profiles were closer to the IDC samples compared to the rest of the 132 ‘HNB’ tissues. Eight of these 11 ONTs had at least 50% of their over-expressed probe sets at a percentile rank (i.e. median percentile rank) greater than 80% among all the 143 ‘HNB’ tissues (i.e., the top 20%). This consistent pattern was seen at 3 and 4 fold-change cutoffs used (FIG. 1). Looking at the median percentile rank of the under-expressed probe sets for these 8 outlier tissues, the inventors found one ONT (T8607A1) with a median percentile rank of 10% for the down-regulated genes in the IDC signature used. In fact this tissue had a very high median percentile rank (94%), for the up-regulated genes in the IDC signature used. Seven other ONTs (N10910A3, N10910A4, N10739D4, N10180C2, N8627A2, N8463I2, and N8380A2) had a median percentile rank between 30% and 48%, for the down-regulated genes. The other 3 of the 11 ONTs had a median percentile rank less than 20% (i.e., the bottom 20%) for the under-expressed probe sets, but two tissues (N11451A4 and N11103G2) had a median percentile rank of 18% and 31% for the up-regulated genes. The other one had a higher median percentile rank, 65%, for up-regulated genes (N11123D3).

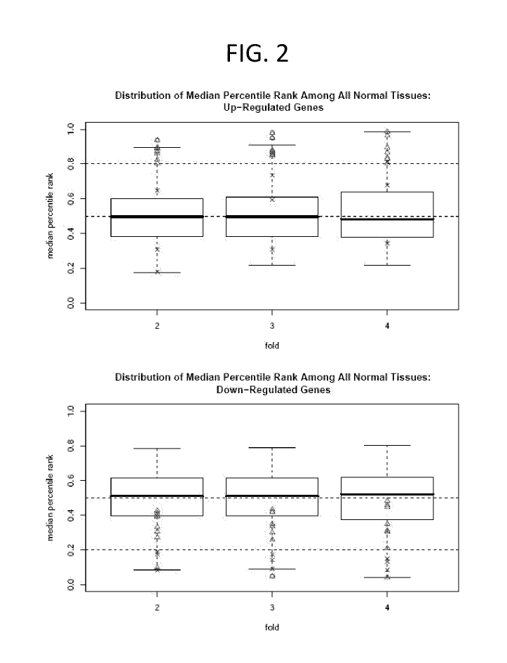

The inventors examined the distribution of the median percentile rank of the up-regulated and the down-regulated genes among all HNBs (including the outlier tissues) using the fold change cutoffs of 2, 3, and 4 to see how the 11 ONTs differ from the rest of the HNBs (n=132). Results showed that the majority of tissues distributed between 40% and 60% and centered around 50% in terms of the median percentile rank either for the up-regulated or the down-regulated genes (FIG. 2). Eight out of 11 outlier tissues had a very high median percentile rank (>80%) for the up-regulated genes. The other three outlier tissues gave a median percentile rank (<20%) for the down-regulated genes.

Table 4 summarizes histological findings of the 11 ONTs. These 11 ONTs were from 10 subjects with two outlier tissues form the same patient. Nine of 10 subjects had cancer developed. The inventors also examined histology of collected adjacent breast tissues (i.e. tissues collected from the same patient) to these 11 outlier tissues. They were 12 histologically normal breast tissues, 6 tissues with cystic change, 1 ALH, and 2 IDC tissues (see Table 1). Gene expression level of these adjacent normal tissues was between the non-adjacent normal breast tissues and the outlier tissues.

TABLE 4
Histological findings in the 11 outlier breast tissues

| Sample | Predominant histological features |
|---|---|
| N8607A1 | Unremarkable breast tissue; no ADH/ALH; no in-situ or invasive |
| | carcinoma. Single duct with mild-moderate epithelial hyperplasia, usual type |
| | (UDH) |
| N11451A4 | Unremarkable breast tissue; no epithelial hyperplasia; no ADH/ALH; no |
| | in-situ or invasive carcinoma |
| N11123D3 | Unremarkable breast tissue; no epithelial hyperplasia; no ADH/ALH; no |
| | in-situ or invasive carcinoma |
| N11103G2 | Unremarkable breast tissue; no epithelial hyperplasia; no ADH/ALH; no |
| | in-situ or invasive carcinoma |
| N10910A4 | Unremarkable breast tissue; no ADH/ALH; no in-situ or invasive |
| | carcinoma. |
| | Fibrocystic changes (20%) |
| N10910A3 | Unremarkable breast tissue; no ADH/ALH; no in-situ or invasive |
| | carcinoma |
| | Fibrocystic changes (20%) |
| N10739D4 | Unremarkable breast tissue; no epithelial hyperplasia; no ADH/ALH; no |
| | in-situ or invasive carcinoma |
| | Occasional focus of sclerosing adenosis, columnar cell change, |
| | microcyst formation |
| N10180C2 | Unremarkable breast tissue; no epithelial hyperplasia; no ADH/ALH; no |
| | in-situ or invasive carcinoma |
| | Rare benign microcyst, mild focal chronic inflammatory infiltrate |
| N8627A2 | Unremarkable breast tissue; no epithelial hyperplasia; no ADH/ALH; no |
| | in-situ or invasive carcinoma |
| N8463I2 | Unremarkable breast tissue; no epithelial hyperplasia; no ADH/ALH; no |
| | in-situ or invasive carcinoma |
| N8380A2 | Unremarkable breast tissue; no epithelial hyperplasia; no ADH/ALH; no |
| | in-situ or invasive carcinoma |

### Example II

### Outlier Gene (OG) Approach

A common set of genes whose expression level is between the normal and IDC tissues was found using a percentile rank approach based on the outlier normal tissues. Those genes with expression percentile rank of greater than 80% (or less than 20%) in at least 70% of outlier normal tissues were selected. The selected IDC genes, outlier tissues, and outlier genes were evaluated by leave-oneo-out cross validation (LOOCV) to assess the reliability.

The OG approach identified a common set of genes whose expression varied (up or down) at high levels in the 11 ONTs. Results showed 109 probe sets (96 unique genes) with up-regulation and 31 probe sets (21 unique genes) with down-regulation in at least 8 ONTs (70%). The level of expression of these genes in the ONTs was between that of HNB and IDCs, suggesting that the change in expression may indicate disease progression.

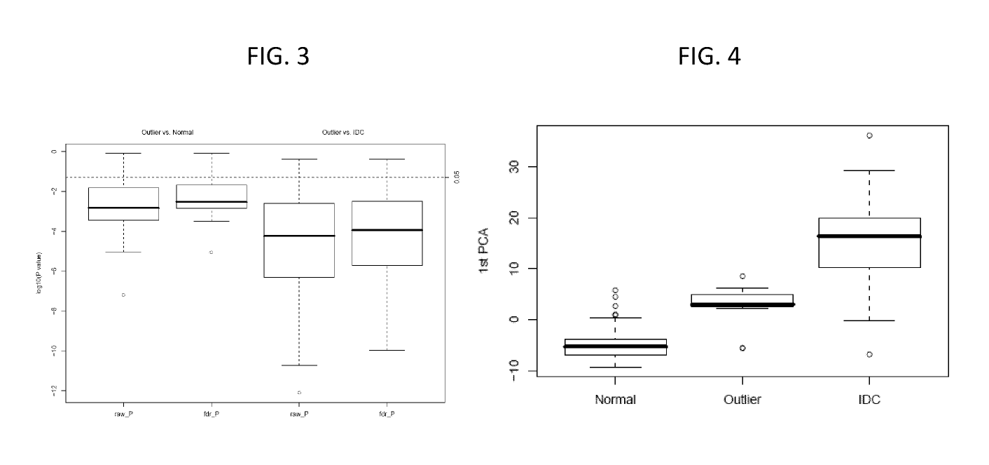

To see how gene expression of ONT is different from HNB and IDC, a two-sample t-test was used to test expression change between (a) HNB versus ONTs and (b) IDC versus ONTs for the outlier genes. False discovery rate (FDR) based on the Benjamini approach was used to adjust p value for simultaneous multiple testing. Results (FIG. 3) showed that 82% of the outlier genes varied significantly between HNB and ONT with an adjusted p<0.05, whereas 94% of the outlier genes varied significantly between IDC and ONT. These results show that expression of the outlier genes was distinct from normal and IDC tissues. In addition, the higher percent of significance level in IDC than in HNB indicated that the expression levels were higher in the IDC tissues than in the normal tissues i.e., expression of the outlier genes in the ONT was close to expression in HBN. This observation is consistent with early stage of tumor development.

Pathway analysis showed that the outlier genes consist primarily of genes that are characteristically identified in growing cells. There were 11 cell cycle pathways with p value <0.01 (Table 5). Most of these genes involved in this pathway were up-regulated. These results were different from the IDC gene signature which had the cell adhesion as the predominant pathway and cell cycle as the second pathway. Since the outlier gene signature was derived from the IDC gene signature, the majority of the outlier genes should be related to cell adhesion. In contrast, the majority of the outlier genes were classified to be primarily associated with DNA replication and mitosis, the two hallmark events associated with proliferation (Table 6).

TABLE 5
Pathway analysis of outlier gene signature with
one predominant cellular processes: cell cycle

| Map | Cell process | p-Value | Genes | |
|---|---|---|---|---|
| The metaphase checkpoint | cell cycle | 3.50E−21 | 14 | 36 |
| Role APC in cell cycle | cell cycle | 1.93E−13 | 11 | 53 |
| regulation | | | | |
| Spindle assembly and | cell cycle | 5.01E−11 | 11 | 86 |
| chromosome separation | | | | |
| Chromosome condensation | cell cycle | 1.11E−10 | 8 | 33 |
| in prometaphase | | | | |
| Role of Nek in cell cycle | cell cycle, protein | 2.23E−07 | 7 | 56 |
| regulation | kinase cascade | | | |
| Nucleocytoplasmic transport | cell cycle | 2.18E−05 | 4 | 22 |
| of CDK/Cyclins | | | | |
| Initiation of mitosis | cell cycle | 3.23E−05 | 5 | 48 |
| Sister chromatid cohesion | cell cycle | 1.44E−04 | 4 | 35 |
| Transition and termination of | cell cycle | 1.61E−04 | 4 | 36 |
| DNA replication | | | | |
| Cell cycle (generic schema) | cell cycle | 1.01E−03 | 3 | 26 |
| ATM/ATR regulation of | cell cycle | 1.40E−03 | 3 | 29 |
| G2/M checkpoint | | | | |

TABLE 6
Outlier genes associated with DNA replication and mitosis

| DNA replication: | Mitosis: |
|---|---|
| Genes Induce at | Genes Induced in |
| G1-S and S-phase | G2 and G2-M |
| TK1 | STK6 |
| CCNE2 | TOP2A |
| MCM2 | BIRC5 |
| MCM4 | CCNB2 |
| PCNA | CDC20 |
| CDCA5 | DLG7 |
| RAD51AP1 | LOC153222 |
| RRM2 | CCNB1 |
| EZH2 | ECT2 |
| MLF1IP | MAPK13 |
| DONSON | BUB1 |
| TYMS | TPX2 |
| SMC2L1 | ANLN |
| SMC4L1 | PBK |
| | BUB1B |
| | FOXM1 |
| | CDCA3 |
| | CKAP2 |
| | NUSAP1 |
| | TTK |
| | KIF23 |
| | MAD2L1 |
| | KIF11 |
| | MELK |
| | DKFZp762E1312 |
| | KNTC2 |
| | CDC2 |

The genes associated with DNA replication include markers associated with origin or replication licensing (MCM2, MCM4), deoxynucleotide synthesis (TK1, RRM2), and cell cycle control (CCNE). Among the mitosis associated genes are well known regulatory proteins that control the spindle checkpoint and chromosome segrartion, including cyclin B1 and B2, aurora kinase A (STK6), budding uninhibited by benzimidazoles 1 BUB1, TOP2A, and CDC2. Importantly, this class of genes exhibits periodic expression at the transcriptional level in cultured cells, and genes related to S-phase and mitosis are also found highly expressed in tumors in cases where there is a relatively high fraction of cycling cells. Many of these genes were identified with multiple probe sets. Taken together, these results show that the outlier gene signature is uniquely distinct from the IDC gene signature and the outlier genes were not randomly identified.

The inventors compared the results using the whole data and the ones by LOOCV in three metrics: IDC genes, outlier tissues, and outlier genes. Analysis of LOOCV yielded a high degree of consistency. Most IDC genes (>98%), outlier tissues (>90%), and outlier genes (>90%) based on the whole data were matched to ones in each leave-one-out step. The inventors further performed principal component analysis (PCA) for the outlier genes in LOOCV to see if the overall expression of the outlier genes shows a progression pattern. This was done by reducing the outlier gene expressions into a summary score based on the first principal component (a linear combination of these gene expressions). Specifically, PCA was used to obtain the first principal component score for all the tissues except the ONTs (i.e., IDC and HNB) at each LOOCV step. The built-up PCA model was used to calculate first PCA for the dropped sample. FIG. 4 shows the summarized score for ONT was between the score of HNB and IDC, indicating disease progression.

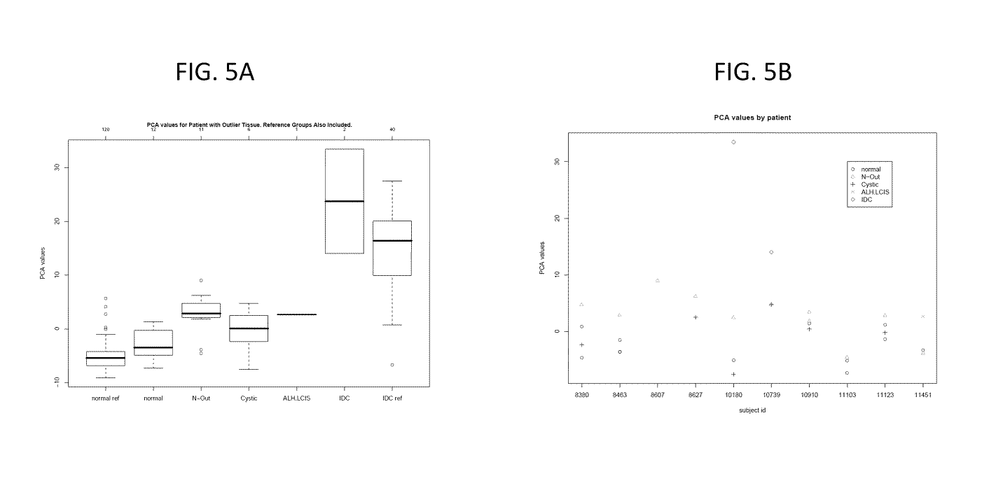

Re-examination of outlier tissues and the adjacent tissues included a PCA model using all IDC and HNB tissues except the ONT and adjacent breast tissues. The first principal component score was then used to represent the summary score for the outlier gene expressions (140 probe sets) at each tissue. The first PCA score for the ONT and the adjacent tissues was estimated based on the PCA model. FIG. 5A shows that, among the normal breast tissues, the median of the first PCA score was highest in the outlier tissues followed by the adjacent normal tissues. The other normal tissues (normal reference) had the lowest score. Interestingly, other adjacent tissues, such as cystic change, ALH, and IDC, had also a higher first PCA score compared to the normal reference. Note that the two adjacent IDCs were relatively higher than the other IDCs.

Furthermore, the inventors compared the first PCA score between the outlier tissues and the adjacent tissues within each subject. FIG. 5B shows majority of the outlier tissues had a higher score than its adjacent normal or cystic change tissues. One outlier tissue (N11451A4) had a lower score than its adjacent normal tissue. Another outlier tissue (N11103G2) had a higher score than its adjacent normal tissue, but was relatively lower than the other normal tissue. Both outlier tissues were classified because they had a lower median percentile rank less than 20% (i.e., the bottom 20%) for the under-expressed probe sets. However, their median percentile rank for the up-regulated genes was also low (18% and 31%) which might explain a lower PCA score.

### Example III

### cDNA Array

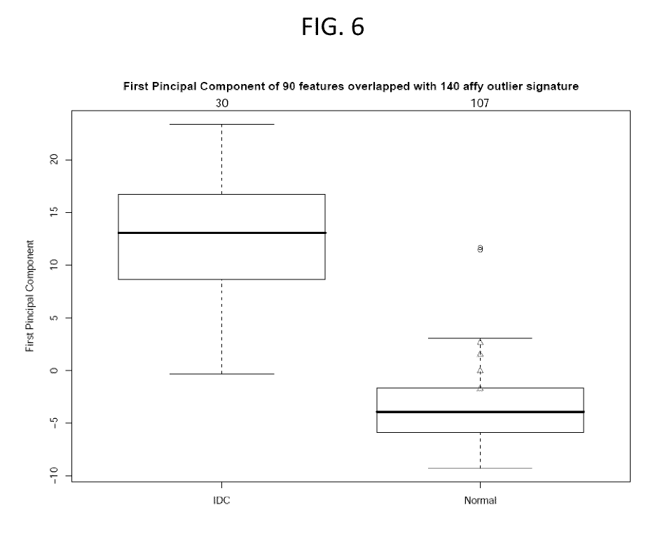

A set of normal breast and IDC samples were collected to establish the validity of the inventive method with a cDNA array. Some normal, IDC, and 4 outlier tissues were analyzed and resulted in 90 overlapping features. The inventors applied PCA to these genes to obtain the 1^{st} PCA score. Results showed the PCA score was higher in IDC than in the normal group (FIG. 6). The four outlier tissues had a relatively higher score (above 75^{th} percentile) compared to the other normal breast tissues.

### Example IV

### Affymetrix Array

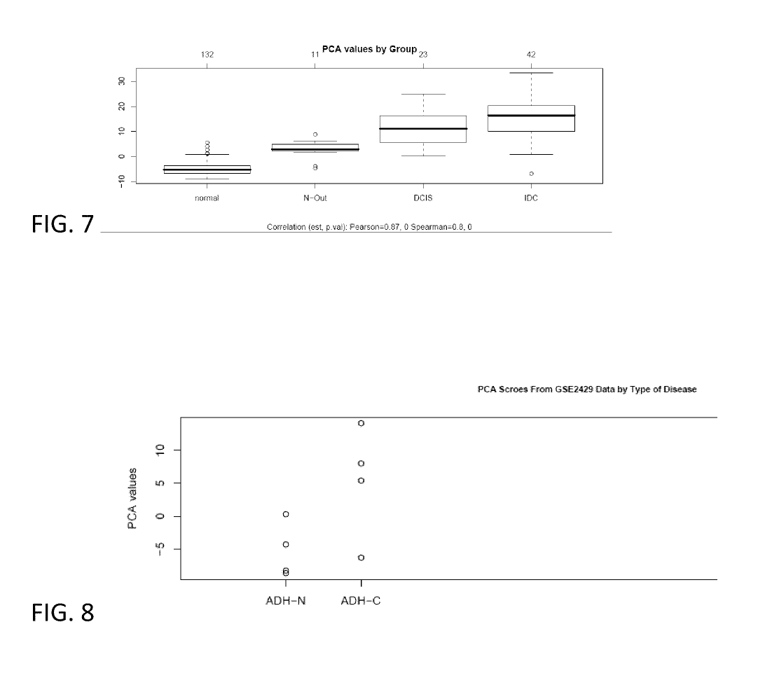

The inventors collected a set of DCIS samples which were processed in Affymetrix platform. Some normal, IDC, and 4 outlier tissues were analyzed. These DCIS samples were used to evaluate the disease progression feature for the outlier gene signature. Based on the PCA model derived from the normal breast and IDC tissues (excluding outlier tissues), the inventors calculated the 1^{st} PCA score for the DCSI tissues. Result showed a clear progression pattern from HNB, outlier, DCSI, to IDC (FIG. 7).

Validation of the ability of the outlier gene signature to predict the risk of cancer development is supported by several studies showing ADH and DCIS as precursors of IDC. Since the outlier gene signature shows a progressive pattern from normal tissue to IDC, with ADH and DCIS as intermediate stages, the utility of the signature to predict the risk of cancer development is further evident.

### Example V

### ADH Study Validation

Poola et al. collected 4 ADH tissues from patients without a history of breast cancer, and another 4 ADH tissues with breast cancer development (the inventors labeled these tissues as ADHC). Microarray experiment was done using Affymetrix U133A chip. Expression data was generated using MASS. Comparison of the gene expression between ADH and ADHC was performed to identify differentially expressed genes. (Nature Medicine 11, 481-483 (2005).

For validation purpose, the inventors transformed the data into log2 scale and normalized the data using quantile method. The inventors limited to a set of 102 probe sets which were overlapped with our 140 outlier probe sets. The inventors applied PCA to these overlapped 102 probe sets to obtain the 1^{st} PCA score for the 4 AHDs and 4 ADHCs. Result showed the ADHC group had a higher PCA score than the ADH group in (FIG. 8). Majority of the ADHC tissues (3 out of 4) yielded a PCA score above 5, in contrast to most AHD tissues with a negative PCA score. This observation indicated the ability of the outlier gene signature to access cancer risk by differentiating AHD tissue between with and without cancer.

### Example VI

### Normal and IDC Study Validation

Turashvili et al. selected 10 patients (5 IDCs and 5 ILCs) and collected one tumor tissue (IDC or ILC) with two normal tissues (ductal or lobular cells) from each subject. Samples were analyzed using Affymetrix U133 Plus 2.0 chip. Data was processed based on RMA method. (Biomed. Papers 149(1), 57-62 (2005)).

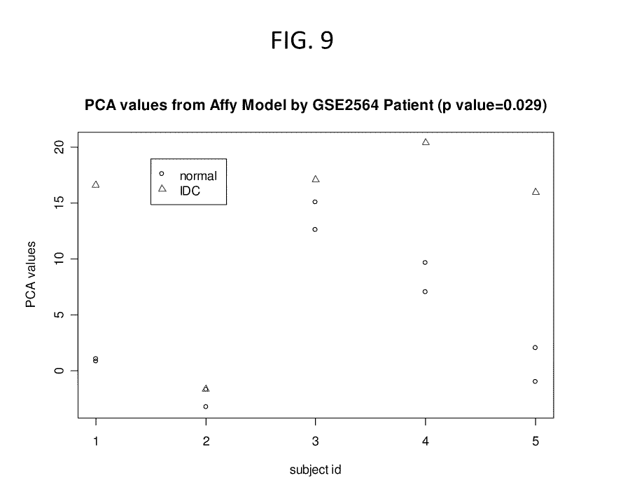

For validation purpose, the inventors used the built-up PCA model from Example IV to predict the 1^{st} PCA score for the 5 IDCs and the associated 10 normal breast tissues. FIG. 9 shows the PCA score was higher in IDC than in normal tissue within the same patient (p value=0.029 based on the random effect model to control for subject variation). This result indicated the outlier gene signature was able to differentiate IDC versus normal tissue.

### Example VII

### Validation Using Data Set of Ma et al.

The inventors used a published data set from Ma et al. to validate (a) the gene signature differentiating between normal and IDC tissues (IDC signature), and most importantly (b) the outlier gene signature. Ma et al. conducted a cDNA microarray study to generate gene expression profiles of the premalignant (ADH), preinvasive (DCIS) and invasive (IDC) stages of human breast cancer. (PNAS May 13, 2003 vol. 100 no. 10 5974-5979).

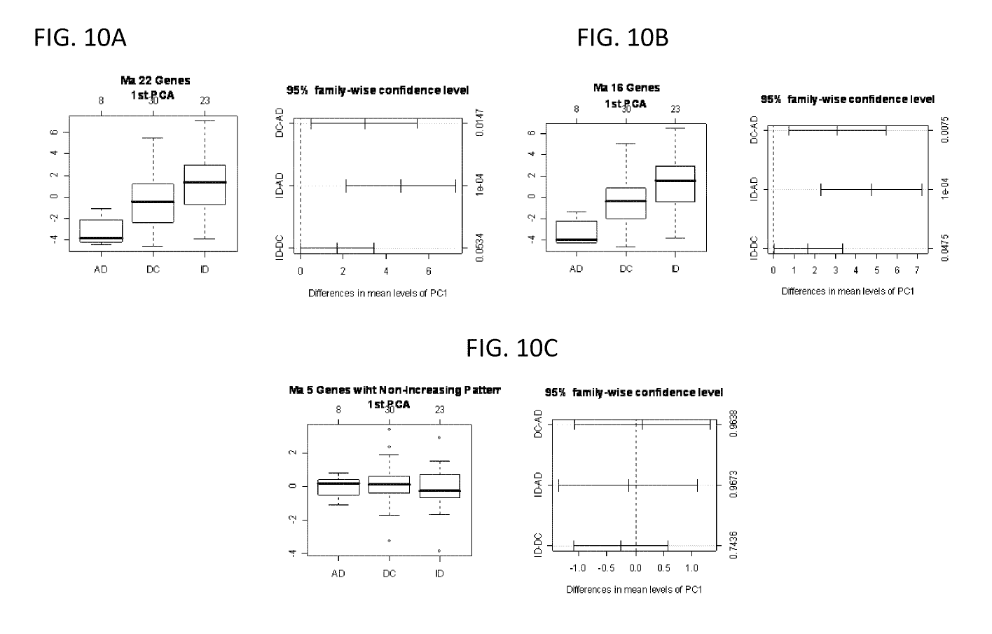

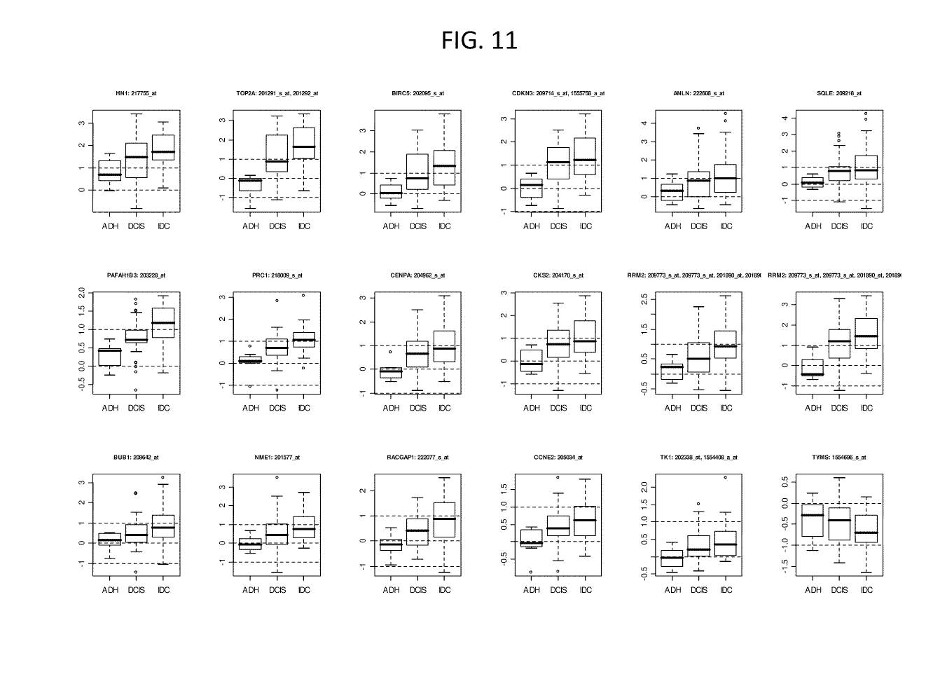

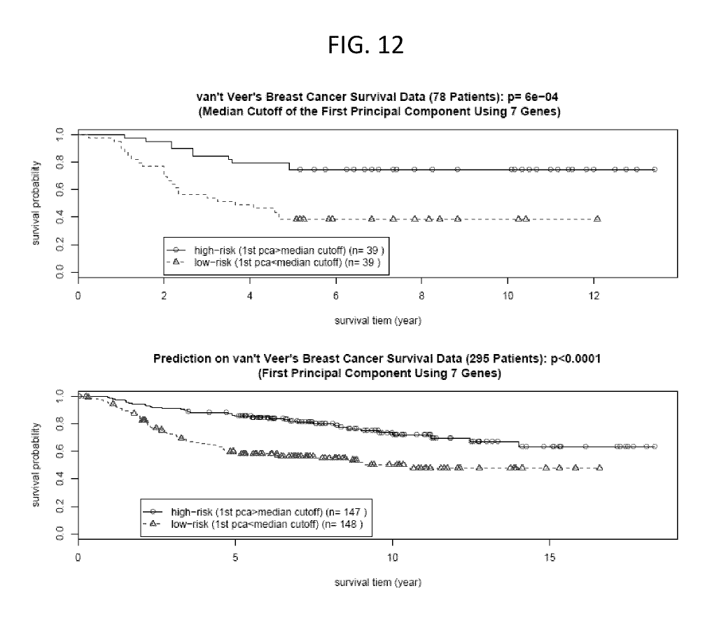

The inventors examined the outlier gene signature (117 unique genes) in the Ma et al. data and found 22 matched genes (21 unique genes with RRM2 gene duplicated). The inventors analyzed these 21 genes using principal component analysis. Results showed an increasing pattern from ADH to IDC in the first principal component score (FIG. 10A). Specifically, the score in ADH group was deviated away from the DCIS and IDC (p value=0.01 and 0.0001). Univariate analysis of these 21 genes also showed a majority of them with a statistically significant fold change (>2). Moreover, 16 genes displayed a similar increasing pattern to the one in our data (FIG. 11).

To see whether the 16 genes dominate PCA analysis results, the inventors compared two sets of genes (16 increasing genes versus 5 non-increasing genes) in PCA analysis. Result shown in FIG. 10B for the 16 increasing genes yielded an enhanced increasing pattern in the 1^{st} principal component score. In contrast, PCA analysis in the 5 non-increasing genes showed that the three groups (ADH, DCIS, and IDC) had a similar distribution of the 1^{st} principal component score around 0 (FIG. 10C). This observation indicated these 16 genes may be potential candidates of the driving force to form the progression pattern. Most of these 16 genes were associated with cell cycle and DNA replication and were periodically expressed in the S/G2/M interval.

Data analysis showed a significant number of overlapping genes between the IDC signature of Ma et al. and the inventor's IDC signature. Specifically, of the 1038 unique genes (inventor's IDC signature), 177 genes matched to the Ma et al. data. Among them, there were 81 significant genes (46%) with q=0 and a fold change >2. Compared to the unmatched genes (1763 genes) which had 302 significant genes (17%), the Fisher exact test showed a strong association of significant genes between the two data sets with a p value <10-15. This validation suggests this gene signature as a molecular hallmark of IDC that can identify IDC when applied to an independent test set of human IDCs.

It will be seen that the advantages set forth above, and those made apparent from the foregoing description, are efficiently attained and since certain changes may be made in the above construction without departing from the scope of the invention, it is intended that all matters contained in the foregoing description or shown in the accompanying drawings shall be interpreted as illustrative and not in a limiting sense.

It is also to be understood that the following claims are intended to cover all of the generic and specific features of the invention herein described, and all statements of the scope of the invention which, as a matter of language, might be said to fall there between.

What is claimed is:

1. A method for determining a prognosis of breast cancer in a breast cancer patient, comprising classifying said patient as having a good prognosis or a poor prognosis using measurements of a plurality of gene products in a histologically normal cell sample taken from said patient, said gene products being respectively products of a plurality of the genes listed in Table 2or respective functional equivalents thereof, wherein said good prognosis predicts survival of a patient within a predetermined time period from obtaining the histologically normal sample from said patient, and said poor prognosis predicts non-survival of a patient within said time period.

2. The method of claim 1, wherein said plurality of gene products are of at least 7 of the genes listed in Table 2.

3. The method of claim 1, further comprising obtaining said marker profile by a method comprising measuring said plurality of gene products in said histologically normal cell sample.

4. The method of claim 1, wherein said time period is between about 2 and 6 years.

5. The method of claim 1, wherein said gene products are products of at least 7 of the genes, respectively, of Table 1.

6. The method of claim 5, wherein said gene products are products of at least 20 of the genes, respectively, of Table 1.

7. The method of claim 1, wherein each of said gene products is a gene transcript.

8. The method of claim 7, wherein measurement of each said gene transcript is obtained by a method comprising contacting a positionally-addressable microarray with nucleic acids from said cell sample or nucleic acids derived therefrom under hybridization conditions, and detecting the amount of hybridization that occurs, said microarray comprising one or more polynucleotide probes complementary to a hybridizable sequence of each said gene transcript.

9. The method of claim 1, wherein each of said plurality of gene products is a protein.

10. A method for evaluating whether a breast cancer patient should be treated with chemotherapy, comprising (a) classifying said patient as having a good prognosis or a poor prognosis using the method of claim 1; and (b) determining that said patient's predicted survival time favors treatment of the patient with chemotherapy if said patient is classified as having a poor prognosis.

11. A microarray comprising for each of a plurality of genes listed in Table 2, one or more polynucleotide probes complementary and hybridizable to a sequence in said gene, wherein polynucleotide probes complementary and hybridizable to said genes constitute at least 50% of the probes on said microarray.

12. The microarray of claim 11, wherein said plurality of genes is at least 7 of the genes of Table 1.

13. The microarray of claim 12, wherein said plurality of genes is at least 20 of the genes of Table 1.
